(19) United States
(12) Patent Application Publication
(10) Pub. No.: US 2013/0317123 A1
(43) Pub. Date: Nov. 28, 2013

(54) LARGE DELETIONS IN HUMAN PMS2 GENE AND USE THEREOF

(71) Applicant: MYRIAD GENETICS, INC., (US)

(72) Inventors: Karla Bowles, Salt Lake City, UT (US); Benoit Leclair, Salt Lake City, UT (US); Saradha Rajamani, Salt Lake City, UT (US); Jeffrey T. Trost, Salt Lake City, UT (US); Jack W. Landon, Salt Lake City, UT (US); Benjamin B. Roa, Salt Lake City, UT (US)

(73) Assignee: MYRIAD GENETICS, INC., Salt Lake City, UT (US)

(21) Appl. No.: 13/849,394

(22) Filed: Mar. 22, 2013

Related U.S. Application Data
(60) Provisional application No. 61/614,850, filed on Mar. 23, 2012.

(51) Int. Cl. C12Q 1/68

(52) U.S. Cl. CPC C12Q 1/6886
USPC 514/789; 435/6.11; 436/501; 435/7.92

(57) ABSTRACT

Large deletions have been identified in the PMS2 gene in patients. The large deletions predispose the patients to Lynch syndrome associated cancers.

### RELATED APPLICATIONS

This application claims priority to U.S. provisional application No. 61/614,850, filed Mar. 23, 2012, the entire contents of which is hereby incorporated by reference.

### TECHNICAL FIELD OF THE INVENTION

This invention generally relates to human genetics, particularly to the identification of genetic polymorphic variations in the human PMS2 gene and methods of identifying or using the identified genetic polymorphisms.

### TECHNICAL BACKGROUND OF THE INVENTION

Lynch syndrome (also known as Hereditary Nonpolyposis Colon Cancer), is an autosomal dominant genetic syndrome that is responsible for 2-4% of colorectal cancers, as well as other cancers, within the United States. Hampel H, et al. Screening for Lynch syndrome (hereditary nonpolyposis colorectal cancer) among endometrial cancer patients. Cancer Res 2006; 66:7810-7817. Lynch syndrome associated cancers include: colon cancer, endometrial cancer, ovarian cancer, stomach cancer, small intestinal cancer, hepatobiliary tract cancer, ureter/renal pelvis cancer, skin cancer (sebaceous tumor), and brain cancer.

Diagnostic analysis of the PMS2 gene is complicated by the presence of multiple highly homologous pseudogenes, which span exons 1-5, 9, and 11-15 of the PMS2 gene. There is a need for identification of novel variants and deletions in the PMS2 gene that predispose individuals for cancer, as well as methods for identifying those variants and deletions.

### SUMMARY OF THE INVENTION

The present invention relates to the discovery of a number of large deletions in human PMS2 gene in patients. A detailed description of the newly discovered deletion mutations is provided in Table 1. These deletions are believed to be deleterious and cause significant alterations in structure or biochemical activities in the PMS2 gene products expressed from mutant PMS2 genes. Patients with such deletions in one of their PMS2 genes are predisposed to, and thus have a significantly increased likelihood of, Lynch syndrome associated cancers. Therefore, these deletion variants are useful in genetic testing as markers for the prediction of predisposition to cancers, including Lynch syndrome associated cancers, and in therapeutic applications for treating cancers.

Accordingly, in a first aspect of the present invention, isolated PMS2 nucleic acids (genomic DNAs, corresponding mRNAs and corresponding cDNAs) are provided comprising one of the newly discovered genetic variants summarized in Table 1 below.

In accordance with another aspect of the invention, isolated polypeptides are provided which are PMS2 protein variants comprising at least a portion of the amino acid sequence of a PMS2 protein. The PMS2 protein variants are encoded by an isolated PMS2 gene sequence of the present invention.

The present invention also provides a method for preparing an antibody to a PMS2 protein variant according to the present invention. Preferably, the antibody prepared in this method is selectively immunoreactive with one or more of the newly discovered PMS2 protein variants.

In accordance with another aspect of the present invention, a variety of methods are provided for predicting a predisposition to cancer in a patient. In one embodiment these methods comprise detecting a deletion in the PMS2 gene. In a related embodiment, an above identified deletion in the PMS2 gene indicates a predisposition to cancer. The detection step used in such methods can involve the analysis of PMS2 genomic DNA, cDNA or polypeptides. Analyses of nucleic acids in these instances can involve amplification-based approaches or hybridization-based approaches. In one embodiment, the deletion is detected using one or more Multiplex Ligation-dependent Probe Amplification (MLPA) assays. Analyses of polypeptides can involve determining whether or not the variant PMS2 polypeptide is truncated, or contains characteristic epitopes that can be specifically detected with an appropriate antibody.

In yet another embodiment of this aspect of the present invention these methods involve detecting specific sequences in PMS2 genomic DNA or cDNA that are formed by the joining of the normally-separated sequences that occur on either side of the deleted region. Detection of these indicative or characteristic nucleic acids in these instances can involve amplification-based approaches or hybridization-based approaches. In one embodiment, the specific sequence is detected using one or more MLPA assays.

In accordance with another aspect of the invention, methods of detecting a mutation in PMS2 nucleic acid are disclosed. In a specific embodiment, the method comprises analyzing PMS2 nucleic acid in a sample obtained from an individual. In one embodiment, the method comprises obtaining a sample from the individual. In one embodiment, the method comprises detecting any one of the mutations in Table 1.

In accordance with another aspect of the invention, methods for diagnosing an individual with Lynch Syndrome are disclosed. In one embodiment, the method comprises analyzing PMS2 nucleic acid in a sample obtained from an individual. In one embodiment, the method comprises obtaining a sample from the individual. In one embodiment, the method comprises detecting any one of the mutations in Table 1. In one embodiment, the method comprises diagnosing the individual with Lynch Syndrome based at least in part on detecting a mutation.

In accordance with another aspect of the invention, methods for prophylactically treating an individual for a Lynch Syndrome associated cancer are disclosed. In one embodiment, the method comprises analyzing PMS2 nucleic acid in a sample obtained from an individual. In one embodiment, the method comprises obtaining a sample from the individual. In one embodiment, the method comprises detecting any one of the mutations in Table 1. In one embodiment, the method comprises treating the individual with a medicament or other intervention to reduce the likelihood of the individual developing a Lynch Syndrome associated caner. In one embodiment, the method comprises performing surgery on the individual to reduce the likelihood of the individual developing a Lynch Syndrome associated caner.

In accordance with another aspect of the invention, a detection kit is also provided for detecting, in an individual, an elevated risk of cancer. In a specific embodiment, the kit is used in determining a predisposition to Lynch syndrome associated cancers. The kit may include, in a partitioned carrier or confined compartment, any nucleic acid probes or primers, or antibodies useful for detecting the PMS2 variants of the present invention as described above. The kit can also include other reagents such as reverse transcriptase, DNA polymerase, buffers, nucleotides and other items that can be used in detecting the genetic variations and/or amino acid variants according to the method of this invention. In one embodiment, the kit may include primers for use in one or more MLPA assays. In addition, the kit preferably also contains instructions for its use.

In any of the foregoing aspects, certain embodiments encompass a Lynch Syndrome associated cancers. In some embodiments, the Lynch Syndrome associated cancers are: colon cancer, endometrial cancer, ovarian cancer, stomach cancer, small intestinal cancer, hepatobiliary tract cancer, ureter/renal pelvis cancer, skin cancer (sebaceous tumor), and brain cancer.

The foregoing and other advantages and features of the invention, and the manner in which the same are accomplished, will become more readily apparent upon consideration of the following detailed description of the invention taken in conjunction with the accompanying examples and drawings, which illustrate preferred and exemplary embodiments.

### BRIEF DESCRIPTION OF THE FIGURES

FIG. 1 shows an overview of the experiments performed in Example 1. Sequencing and large rearrangement analyses of the MLH1, MSH2, MSH6, PMS2, and EPCAM genes were performed on patient DNA. Approximately 14% of disease-associated mutations identified were found in the PMS2 gene.

FIG. 2 depicts the percentage of Lynch syndrome mutations by gene. 7. Disease-associated mutations were identified in multiple DNA mismatch repair genes. Approximately 37% of causative Lynch syndrome mutations were identified in MSH2, 29% in MLH1, 18% in MSH6, 14% in PMS2, and 1.8% in EPCAM. An additional 1% of mutations detected extended from the 3′ untranslated region of EPCAM through the 5′ end of MSH2.

FIG. 3 is an MLPA plot providing an example of the experimental results. The plot demonstrates a heterozygous duplication of PMS2 exons 11 and 12. Due to the presence of a cross-reacting pseudogene (ψ), the exon 12 MLPA probe normally detects 4 alleles (2 coding and 2 pseudogene). The duplication results in the presence of a 5th allele and a corresponding ˜25% increase in signal strength. Other exon 11 and 12 probes are specific to either the pseudogene (2 copies present) or the coding gene, which is present in 3 copies in this patient and shows a corresponding ˜50% increase in signal strength.

FIG. 4 is an overview of the PMS2 gene structure. The PMS2 gene is composed of 15 exons. Multiple pseudogenes spanning exons 1-5, 9, and 11-15 have been identified elsewhere in the genome. However, the interpretation of large rearrangement analysis results is significantly impacted for exons 11-15. We have identified 14 unique large rearrangements within this gene that are associated with increased cancer risk.

### DETAILED DESCRIPTION OF THE INVENTION

### 1. Definitions

The terms “genetic variant,” “mutation,” and “nucleotide variant” are used herein interchangeably to refer to changes or alterations to a reference PMS2 gene sequence at a particular locus, including, but not limited to, nucleotide base deletions, insertions, inversions, and substitutions in the coding and noncoding regions. Deletions may be of a single nucleotide, a portion or a region of the nucleotide sequence of the gene, or of the entire gene sequence. Insertions may be of one or more nucleotides. The genetic variants may occur in transcriptional regulatory regions, untranslated regions of mRNA, exons, introns, or exon/intron junctions. The genetic variants may or may not result in stop codons, frame shifts, deletion of amino acids, altered amino acid sequence, or altered protein expression level. The mutations or genetic variants can be somatic, i.e., occur only in certain tissues of the body and are not inherited in the germline, or germline mutations, i.e., inherited mutations found in all tissues.

“Genetic polymorphism” as used herein refers to the phenomena that two or more genetic variants in a particular locus of a gene are found in a population.

The term “allele” or “gene allele” is used herein to refer generally to a naturally occurring gene having the reference sequence or a gene containing a specific genetic variant.

As used herein, the term “PMS2 nucleic acid” means a nucleic acid molecule the nucleotide sequence of which is found uniquely in a PMS2 gene or a substantially equivalent form thereof. That is, the nucleotide sequence of a “PMS2 nucleic acid” can be a full-length sequence of, or a portion found in, either PMS2 genomic DNA or mRNA/cDNA, either wild-type or naturally existing variant PMS2 gene, or an artificial nucleotide sequence encoding a wild-type PMS2 protein or naturally existing polymorphic variant PMS2 protein. In some embodiments, PMS2 nucleic acid comprises a nuclide acid that codes for UniProt P54278. In some embodiments, PMS2 means the gene identified as HGNC: 91221, Entrez Gene: 53952, Ensembl: ENSG000001225127, NCBI Reference Sequence: NG_{—}008466.1 or OMIM: 600259.

The term “PMS2 nucleic acid variant” refers to a naturally existing PMS2 nucleic acid comprising at least one nucleotide variant.

As used herein, the term “amino acid variant” refers to amino acid changes to a reference PMS2 protein sequence resulting from nucleotide variants or mutations to the reference gene encoding the reference PMS2 protein. The term “amino acid variant” is intended to encompass not only single amino acid substitutions, but also amino acid deletions, insertions, and other significant changes of amino acid sequence in a PMS2 protein, such as truncations.

The term “PMS2 protein variant” is used herein relative to a reference PMS2 protein to mean a PMS2 protein found in a population that is the coding product of a PMS2 gene allele containing genetic variants such as single nucleotide substitutions, insertions, deletions, and DNA rearrangements, which lead to alterations in the protein sequence of the protein variant.

The term “locus” refers to a specific position or site in a nucleotide sequence of a gene, or amino acid sequence of a protein. Thus, there may be one or more contiguous nucleotides in a particular gene locus, or one or more amino acids at a particular locus in a polypeptide. Moreover, “locus” may also be used to refer to a particular position in a gene sequence where one or more nucleotides have been deleted, inserted, or inverted.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, etc. Modifications also include intra-molecular crosslinking and covalent attachment of various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

The terms “primer,” “probe,” and “oligonucleotide” may be used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated apart by denaturation. Normally, they have a length of from about 8 nucleotides to about 200 nucleotides, preferably from about 12 nucleotides to about 100 nucleotides, and more preferably about 18 to about 50 nucleotides. They can be labeled with detectable markers or modified in any conventional manners for various molecular biological applications.

The term “isolated,” when used in reference to nucleic acids (which include gene sequences or fragments) of this invention, is intended to mean that a nucleic acid molecule is present in a form other than found in nature in its original environment with respect to its association with other molecules. For example, since a naturally existing chromosome includes a long nucleic acid sequence, an “isolated nucleic acid” as used herein means a nucleic acid molecule having only a portion of the nucleic acid sequence in the chromosome but not one or more other portions present on the same chromosome. Thus, for example, an isolated gene typically includes no more than 25 kb of naturally occurring nucleic acid sequence which immediately flanks the gene in the naturally existing chromosome or genomic DNA. However, it is noted that an “isolated nucleic acid” as used herein is distinct from a clone in a conventional library such as genomic DNA library and cDNA library in that the clones in a library are still in admixture with almost all the other nucleic acids in a chromosome or a cell. An isolated nucleic acid can be in a vector.

The term “isolated nucleic acid” embraces “purified nucleic acid” which means a specified nucleic acid is in a substantially homogenous preparation of nucleic acid substantially free of other cellular components, other nucleic acids, viral materials, or culture medium, or chemical precursors or by-products associated with chemical reactions for chemical synthesis of nucleic acids. In some embodiments, the isolated nucleic acid is removed from a larger nucleic acid molecule by cleavage of chemical bonds to surrounding nucleotides. In some embodiments, the isolated nucleic acid is no more than 25 kb. In some embodiments, the isolated nucleic acid is no more than 15 kb. In some embodiments, the isolated nucleic acid is no more than 10 kb. In some embodiments, the isolated nucleic acid is no more than 8 kb. In some embodiments, the isolated nucleic acid is no more than 5 kb. Typically, a “purified nucleic acid” can be obtained by standard nucleic acid purification methods. In a purified nucleic acid, preferably the specified nucleic acid molecule constitutes at least 15 percent of the total nucleic acids in the preparation. The term “purified nucleic acid” also means nucleic acids prepared from a recombinant host cell (in which the nucleic acids have been recombinantly amplified and/or expressed), or chemically synthesized nucleic acids.

The term “isolated nucleic acid” also encompasses a “recombinant nucleic acid” which is used herein to mean a hybrid nucleic acid produced by recombinant DNA technology having the specified nucleic acid molecule covalently linked to one or more nucleic acid molecules that are not the nucleic acids naturally flanking the specified nucleic acid. Typically, such nucleic acid molecules flanking the specified nucleic acid are no more than 50 kb. In some embodiments, the specified nucleic acid molecule covalently linked to the one or more flanking nucleic acids is no more than 25 kb. In some embodiments, the specified nucleic acid molecule covalently linked to the one or more flanking nucleic acids is no more than 15 kb. In some embodiments, the specified nucleic acid molecule covalently linked to the one or more flanking nucleic acids is no more than 10 kb. In some embodiments, the specified nucleic acid molecule covalently linked to the one or more flanking nucleic acids is no more than 8 kb. In some embodiments, the specified nucleic acid molecule covalently linked to the one or more flanking nucleic acids is no more than 5 kb. In addition, the specified nucleic acid may have a nucleotide sequence that is identical to a naturally occurring nucleic acid, or a modified form, or mutant form thereof having one or more mutations such as nucleotide substitution, deletion/insertion, inversion, and the like.

In addition, “isolated nucleic acid” further includes a chemically synthesized nucleic acid having a naturally occurring nucleotide sequence or an artificially modified form thereof (e.g., dideoxy forms).

The term “isolated polypeptide” as used herein means a polypeptide molecule is present in a form other than found in nature in its original environment with respect to its association with other molecules. The term “isolated polypeptide” encompasses a “purified polypeptide” which is used herein to mean that a specified polypeptide is in a substantially homogenous preparation, substantially free of other cellular components, other polypeptides, viral materials, or culture medium, or when the polypeptide is chemically synthesized, substantially free of chemical precursors or by-products associated with the chemical synthesis. For a purified polypeptide, preferably the specified polypeptide molecule constitutes at least 15 percent of the total polypeptide in the preparation. A “purified polypeptide” can be obtained from natural or recombinant host cells by standard purification techniques, or by chemical synthesis.

The term “isolated polypeptide” also encompasses a “recombinant polypetide,” which is used herein to mean a hybrid polypeptide produced by recombinant DNA technology or chemical synthesis having a specified polypeptide molecule covalently linked to one or more polypeptide molecules which do not naturally link to the specified polypeptide.

As used herein, “haplotype” is a combination of genetic (nucleotide) variants in a region of an mRNA or a genomic DNA on a chromosome found in an individual. Thus, a haplotype includes a number of genetically linked polymorphic variants that are typically inherited together as a unit.

The term “reference sequence” refers to a polynucleotide or polypeptide sequence known in the art, including those disclosed in publicly accessible databases (e.g., GenBank), or a newly identified gene sequence, used simply as a reference with respect to the variants provided in the present invention. The nucleotide or amino acid sequence in a reference sequence is contrasted to the alleles disclosed in the present invention having newly discovered nucleotide or amino acid variants.

The terms “crossing-over” and “crossover,” are used interchangeably herein, to refer to the reciprocal exchange of material between chromosome homologs—by breakage and reunion—that occurs during meiosis and is responsible for genetic recombination. The term “unequal crossover,” as used herein, refers to a crossover event occurring between homologous sequences in paired chromosome homologs that are not perfectly aligned, or, more generally, describes a recombination event in which the two recombining sites lie at non-identical locations in the two parental DNA molecules. The products of an unequal crossover are two chromosomes, or more generally two progeny DNA molecules, one of which bears a deletion, and the other of which bears a duplication of the nucleotide sequence residing between the mispaired homologous sequences or recombining sites.

### 2. Nucleotide and Amino Acid Variants

In accordance with the present invention, analysis of the nucleotide sequence of genomic DNA corresponding to the PMS2 genes of specific human patients has led to the discovery of a number of mutant PMS2 alleles that exhibit large deletions relative to the reference sequence provided by NCBI Reference Sequence: NG_{—}008466.1. Specifically, fourteen different genetic variants exhibiting large deletions have been discovered. These fourteen different genetic variants, are summarized in Table 1. Of these fourteen different genetic variants corresponding to fourteen different large deletions of nucleotide sequence within the PMS2 gene, identification of at least six was complicated by the presence of highly homologous pseudogenes.

TABLE 1
GENETIC VARIANTS OF THE PMS2 GENE

| Interpretation Complicated | Interpretation Not Complicated |
|---|---|
| By Pseudogenes | By Pseudogenes |
| del exons 1-11 | del exons 1-5 |
| del exons 6-15 | del exons 1-9 |
| del exons 7-11 | del exons 1-10 |
| del exon 11 | del exons 5-9 |
| dup exons 11-12 | del exons 6-9 |
| del entire PMS2 gene | del exon 8 |
| | del exons 9-10 |
| | del exon 10 |

In further accordance with the present invention, the large deletions described in Table 1 were found in patients with a personal and/or family history suggestive of Lynch syndrome or hereditary colon cancer.

### 3. PMS2 Nucleic Acids

In a first aspect of the present invention, isolated nucleic acids are provided comprising a nucleotide sequence of a PMS2 nucleic acid variant identified in accordance with the present invention. The nucleotide sequence is at least 12, 13, 14, 15, 17, 18, 19, 20, 25, 30, or 35 contiguous nucleotides spanning the deletion locus in one of the mutant PMS2 genomic DNAs having one of the deletions in Table 1, or the analogous deletion locus in one of the mutant PMS2 mRNAs, or cDNAs prepared therefrom, expressed from the mutant PMS2 genomic DNAs having one of the deletions. The nucleic acid molecules can be in a form of DNA, RNA, or a chimeric or hybrid thereof, and can be in any physical structures including a single-strand or double-strand or in the form of a triple helix.

In one embodiment, an isolated PMS2 nucleic acid is an oligonucleotide, primer or probe comprising a contiguous span of the nucleotide sequence of a mutant PMS2 sequence (either genomic DNA or cDNA or mRNA sequence) provided in accordance with the present invention. In some embodiments, the oligonucleotide spans a cDNA deletion locus resulted from one of the deletions in Table 1. The oligonucleotide, primer or probe contains at least 12, preferably from about 15, 18, 20, 22, 25, 30, or 40 to about 50, 60, 70, 80, 90, or 100, and more preferably from about 30 to about 50 nucleotides. In one embodiment, the oligonucleotides, primers and probes are specific to a PMS2 nucleic acid variant of the present invention. That is, they selectively hybridize, under stringent conditions generally recognized in the art, to a PMS2 nucleic acid variant of the present invention, but do not substantially hybridize to a reference PMS2 nucleic acid sequence, or to a homologous PMS2 pseudogene sequence under stringent conditions. Such oligonucleotides will be useful in hybridization-based methods, or alternatively amplification-based methods, for detecting the nucleotide variants of the present invention as described in detail below. A skilled artisan would recognize various stringent conditions that enable the oligonucleotides of the present invention to differentiate between a reference PMS2 gene sequence and an isolated PMS2 nucleic acid variant of the present invention. For example, the hybridization can be conducted overnight in a solution containing 50% formamide, 5×SSC, pH7.6, 5×Denhardt's solution, 10% dextran sulfate, and 20 microgram/ml denatured, sheared salmon sperm DNA. The hybridization filters can be washed in 0.1×SSC at about 65° C.

In a related embodiment, the oligonucleoides may have a sequence identical to a sequence found in an exon of the PMS2 gene. In another related embodiment, two related oligonucleotides may be provided for ligation based assays. In embodiments comprising related nucleotides for ligation based assays, two related oligonucleotides have sequences which are respectively identical to sequences in an exon of the PMS2 gene, such that if the two related oligonucleotides hybridize to the complementary strand of a PMS2 nucleic acid, the 5′ end of one of the oligonucleotides will be adjacent to the 3′ end of the other oligonucleotide, such that a ligase may covalently bind the two oligonucleotides together.

The oligonucleotide primers or probes of the present invention can have a detectable marker selected from, e.g., radioisotopes, fluorescent compounds, enzymes, or enzyme co-factors operably linked to the oligonucleotide. The primers, probes and oligonucleotide sequences of the present invention are useful in genotyping and haplotyping as will be apparent from the description below.

It should be understood that any nucleic acid molecules containing a PMS2 sequence described herein are within the scope of this invention. For example, a hybrid nucleic acid molecule may be provided having a PMS2 sequence as described herein operably linked to a non-PMS2 sequence such that the hybrid nucleic acid encodes a hybrid protein having a mutant PMS2 peptide sequence. In another embodiment, a hybrid nucleic acid molecule will be provided having a PMS2 sequence as described herein linked to a primer hybridization sequence. In another embodiment, the present invention provides a vector construct containing one of the nucleic acid molecules of the present invention. As will be apparent to skilled artisans, the vector may be employed to amplify a nucleic acid molecule of the present invention that is contained in the vector construct. Alternatively, the vector construct may be used in expressing a polypeptide encoded by a nucleic acid molecule of the present invention that is contained in the vector construct. Generally, the vector construct may include a promoter operably linked to an isolated nucleic acid molecule (including a full-length sequence or a fragment thereof in the 5′ to 3′ direction or in the reverse direction for the purpose of producing antisense nucleic acids), an origin of DNA replication for the replication of the vectors in host cells and a replication origin for the amplification of the vectors in, e.g., E. coli, and selection marker(s) for selecting and maintaining only those host cells harboring the vectors. Additionally, the vectors preferably also contain inducible elements, which function to control the expression of the isolated gene sequence. Other regulatory sequences such as transcriptional termination sequences and translation regulation sequences (e.g., Shine-Dalgarno sequence) can also be included. An epitope tag coding sequence for detection and/or purification of the encoded polypeptide can also be incorporated into the vector construct. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies to many epitope tags are generally commercially available. The vector construct can be introduced into the host cells or organisms by any techniques known in the art, e.g., by direct DNA transformation, microinjection, electroporation, viral infection, lipofection, biolystics (gene gun), and the like. The vector construct can be maintained in host cells in an extrachromosomal state, i.e., as self-replicating plasmids or viruses. Alternatively, the vector construct can be integrated into chromosomes of the host cells by conventional techniques such as selection of stable cell lines or site-specific recombination. The vector construct can be designed to be suitable for expression in various host cells, including but not limited to bacteria, yeast cells, plant cells, insect cells, and mammalian and human cells. A skilled artisan will recognize that the designs of the vectors can vary with the host used.

In another embodiment, a PMS2 nucleic acid of the present invention is incorporated in a microchip or microarray, or other similar structures. The microarray will allow rapid genotyping and/or haplotyping in a large scale. As is known in the art, in microchips, a large number of different nucleic acids are attached or immobilized in an array on a solid support, e.g., a silicon chip or glass slide. Target nucleic acid sequences to be analyzed can be contacted with the immobilized nucleic acids on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). The microchip technologies combined with computerized analysis tools allow large-scale high throughput screening. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

In a preferred embodiment, a microarray is provided comprising a plurality of the nucleic acids of the present invention such that the nucleotide identity at each of the genetic variant sites disclosed in Table I can be determined in one single microarray.

### 4. PMS2 Polypeptides

The present invention also provides isolated polypeptides having a novel amino acid sequence of a PMS2 protein variant identified in accordance with the present invention. The amino acid sequence may comprise a contiguous sequence of at least 3, 4, 5, 6, 7, 8, 9, 10, 12, or 13 amino acids spanning the deletion locus resulted from deletions identified in Table 1. In some embodiments, the amino acid sequence is at most 13, 15, 20, 50, 100, 200, 300, 400, 500, 1000, 2000 or 5000 amino acids.

In another embodiment, the PMS2 peptides comprise the translation product of any of the PMS2 nucleic acids described herein.

### 5. Antibodies

The present invention also provides antibodies selectively immunoreactive with an isolated PMS2 protein variant of the present invention. As used herein, the term “antibody” encompasses both monoclonal and polyclonal antibodies that fall within any antibody classes, e.g., IgG, IgM, IgA, etc. The term “antibody” also includes antibody fragments including, but not limited to, Fab and F(ab′)₂, conjugates of such fragments, and single-chain antibodies that can be made in accordance with U.S. Pat. No. 4,704,692, which is incorporated herein by reference. Specifically, as used herein, the phrase “selectively immunoreactive with an isolated PMS2 protein variant of the present invention” means that the immunoreactivity of the antibody of the present invention with a PMS2 protein variant of the present invention is substantially higher than that with a PMS2 protein heretofore known in the art so that the binding of the antibody to the protein variant of the present invention is readily distinguishable from the binding of the antibody to the PMS2 protein known in the art based on the strength of the binding affinities. Preferably, the binding constant differs by a magnitude of at least 2 fold, more preferably at least 5 fold, even more preferably at least 10 fold, and most preferably at least 100 fold.

To make the antibody, a PMS2 protein variant of the present invention, or a suitable fragment thereof, can be used to immunize an animal. The PMS2 protein variant can be made by any methods known in the art, e.g., by recombinant expression or chemical synthesis. Preferably, the mutant PMS2 protein fragment consists of less than 100 amino acids, more preferably less than 50 amino acids, and even more preferably less than 25 amino acids. As a result, a greater portion of the total antibodies may be selectively immunoreactive with a PMS2 protein variant of the present invention. Techniques for immunizing animals for the purpose of making polyclonal antibodies are generally known in the art. See Harlow and Lane, Antibodies: A Laboratory Manual, Cold Spring Harbor Laboratory, Cold Spring Harbor, N.Y., 1988. A carrier may be necessary to increase the immunogenicity of the polypetide. Suitable carriers known in the art include, but are not limited to, liposome, macromolecular protein or polysaccharide, or combination thereof. Preferably, the carrier has a molecular weight in the range of about 10,000 to 1,000,000. The polypeptide may also be administered along with an adjuvant, e.g., complete Freund's adjuvant.

The antibodies of the present invention preferably are monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No. 4,376,110, which is incorporated herein by reference. Essentially, B-lymphocytes producing a polyclonal antibody against a protein variant of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein variant of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific desired protein may then be isolated from the library. The isolated cDNA can be introduced into a suitable host cell for the expression of the protein. Thus, recombinant techniques can be used to recombinantly produce specific native antibodies, hybrid antibodies capable of simultaneous reaction with more than one antigen, chimeric antibodies (e.g., the constant and variable regions are derived from different sources), univalent antibodies which comprise one heavy and light chain pair coupled with the Fc region of a third (heavy) chain, Fab proteins, and the like. See U.S. Pat. No. 4,816,567; European Patent Publication No. 0088994; Munro, Nature, 312:597 (1984); Morrison, Science, 229:1202 (1985); Oi et al., BioTechniques, 4:214 (1986); and Wood et al., Nature, 314:446-449 (1985), all of which are incorporated herein by reference. Antibody fragments such as Fv fragments, single-chain Fv fragments (scFv), Fab′ fragments, and F(ab′)₂ fragments can also be recombinantly produced by methods disclosed in, e.g., U.S. Pat. No. 4,946,778; Skerra & Plückthun, Science, 240:1038-1041 (1988); Better et al., Science, 240:1041-1043 (1988); and Bird, et al., Science, 242:423-426 (1988), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv. Immunol., 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. See e.g., U.S. Pat. Nos. 5,530,101; 5,545,806; 6,075,181; PCT Publication No. WO 94/02602; Green et. al., Nat. Genetics, 7: 13-21 (1994); and Lonberg et al., Nature 368: 856-859 (1994), all of which are incorporated herein by reference. The transgenic non-human host animal may be immunized with suitable antigens such as a protein variant of the present invention to illicit specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library that is subsequently screened for clones encoding humanized antibodies with desired binding specificities. In addition, antibodies may also be produced in transgenic plants containing recombinant nucleic acids encoding antibodies.

In accordance with another embodiment of the present invention, a protein microchip or microarray is provided having (1) a PMS2 protein variant of the present invention or a fragment thereof; and/or (2) an antibody selectively immunoreactive with a PMS2 protein variant of the present invention.

Protein microarrays are becoming increasingly important in both proteomics research and protein-based detection and diagnosis of diseases. The protein microarrays in accordance with the present invention will be useful in a variety of applications including, e.g., high throughput screening for compounds capable of modulating the activities of a PMS2 protein variant of the present invention. The protein microarrays are also useful in detecting the mutant PMS2 proteins, and thus can be used in determining a predisposition to cancer, particularly Lynch syndrome associated cancers.

The protein microarray of the present invention can be prepared by a number of methods known in the art. An example of a suitable method is that disclosed in MacBeath and Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phophate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer which functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, asparate, and glutamate residues on the BSA are activated and can form covalent urea or amide linkages with protein samples spotted on the slides. See MacBeath and Schreiber, Science, 289:1760-1763 (2000).

Another example of useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure a uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarray of the present invention can also be made by the method disclosed in PCT Publication No. WO 99/36576 assigned to Packard Bioscience Company, which is incorporated herein by reference. For example, a three-dimensional hydrophilic polymer matrix, i.e., a gel, is first deposited on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Alternatively, the proteins and protein complexes of the present invention can be incorporated into a commercially available protein microchip, e.g., the ProteinChip System from Ciphergen Biosystems Inc., Palo Alto, Calif. The ProteinChip System comprises metal chips having a treated surface that interact with proteins. Basically, a metal chip surface is coated with a silicon dioxide film. The molecules of interest such as proteins and protein complexes can then be attached covalently to the chip surface via a silane coupling agent.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

### 6. Genotyping

In another aspect of the present invention, methods are provided for predicting, in an individual, the likelihood of developing cancer. As described above, the large deletions in PMS2 genes identified in accordance with the present invention are deleterious and predispose individuals having the deletions to cancer, including Lynch syndrome associated cancers. Thus, by detecting, in an individual, the presence or absence of one or more of the PMS2 variants of the present invention, one can reasonably predict a predisposition to cancer, including Lynch syndrome associated cancers.

Numerous techniques for detecting genetic variants are known in the art and can all be used for the method of this invention. The techniques can be nucleic acid-based or protein-based. In either case, the techniques used must be sufficiently sensitive so as to accurately detect the nucleotide or amino acid variations. Very often, a probe is utilized which is labeled with a detectable marker. Unless otherwise specified in a particular technique described below, any suitable marker known in the art can be used, including but not limited to, radioactive isotopes, fluorescent compounds, biotin which is detectable using strepavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a DNA-based detection method, a target DNA sample, i.e., a sample containing PMS2 gene sequence should be obtained from the individual to be tested. Any tissue or cell sample containing the PMS2genomic DNA or mRNA, or a portion thereof, can be used. Preferably, a tissue sample containing cell nuclei and thus genomic DNA can be obtained from the individual. Blood samples can also be useful, except that only white blood cells and other lymphocytes have cell nuclei, while red blood cells are enucleated and contain mRNA. Nevertheless, mRNA is also useful as it can be analyzed for the presence of nucleotide variants in its sequence or serve as template for cDNA synthesis. The tissue or cell samples can be analyzed directly without much processing. Alternatively, nucleic acids including the target PMS2 nucleic acids can be extracted, purified, or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of the deletion mutations identified in the present invention, one technique is simply sequencing the target PMS2 genomic DNA or cDNA, particularly the region spanning the deletion locus to be detected. Various sequencing techniques are generally known and widely used in the art including the Sanger method and the Gilbert chemical method. The newly developed pyrosequencing method monitors DNA synthesis in real time using a luminometric detection system. Pyrosequencing has been shown to be effective in analyzing genetic polymorphisms such as single-nucleotide polymorphisms and can also be used in the present invention. See Nordstrom et al., Biotechnol. Appl. Biochem., 31(2):107-112 (2000); Ahmadian et al., Anal. Biochem., 280:103-110 (2000). For example, sequencing primers can be designed based on either mutant or wild-type PMS2 gene intronic or exonic sequences such that the primers have the nucleotide sequence adjacent to a deletion locus identified in accordance with the present invention. In another example, PCR primers are designed based on either mutant or wild-type PMS2 gene intronic or exonic sequences such that PCR amplification generates a PMS2 DNA fragment spanning the deletion locus. As the large deletions identified in accordance with the present invention alter the size of the PMS2 genomic DNA or cDNA, the presence or absence of a deletion mutation according to the present invention can be determined based on the molecular weight of the PCR amplification products generated using the PCR primers. Optionally, DNA sequencing is then performed on the amplified fragment to determine the nucleotide sequence of the suspect region.

Alternatively, the restriction fragment length polymorphism (RFLP) method may also prove to be a useful technique. In particular, the large deletions identified in accordance with the present invention result in the elimination and creation of restriction enzyme recognition sites. Digestion of the mutant PMS2 genomic DNAs or cDNAs with appropriate restriction enzyme(s) will generate restriction fragment length patterns distinct from those generated from wild-type PMS2 genomic DNA or cDNA. Thus, the large deletions in PMS2 of the present invention can be detected by RFLP. The application of the RFLP techniques known in the art to the present invention will be apparent to skilled artisans.

Similarly, genomic DNA can be obtained from a patient sample and digested by appropriate restriction enzyme(s). Southern blot can be performed using a probe having a wild-type PMS2 sequence that is missing from one or more of the PMS2 genetic variants of the present invention. Alternatively, probes specific to the mutant PMS2 nucleic acids of the present invention can also be used.

The presence or absence of a PMS2 deletion mutation identified according to the present invention can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA or mRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present invention is the so-called “oligonucleotide ligation assay” (OLA), in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a mutation at a particular locus in the PMS2 gene, two oligonucleotides can be synthesized, one having the PMS2 sequence just 5′ upstream from the locus with its 3′ end nucleotide being identical to the nucleotide in the mutant locus of the PMS2 gene, the other having a nucleotide sequence matching the PMS2 sequence immediately 3′ downstream from the locus in the PMS2 gene. The oligonucleotides can be labeled for the purpose of detection. Upon hybridizing to the target PMS2 gene under a stringent condition, the two oligonucleotides are subjected to ligation in the presence of a suitable ligase. The ligation of the two oligonucleotides would indicate that the target DNA has a nucleotide variant at the locus being detected.

Detection of the genetic variations identified in accordance with the present invention can also be accomplished by a variety of hybridization-based approaches. Allele-specific oligonucleotides are useful. See Conner et al., Proc. Natl. Acad. Sci. USA, 80:278-282 (1983); Saiki et al, Proc. Natl. Acad. Sci. USA, 86:6230-6234 (1989). Oligonucleotide probes hybridizing specifically to a PMS2 gene allele having a particular gene variant at a particular locus but not to other alleles can be designed by methods known in the art. The probes can have a length of, e.g., from 10 to about 50 nucleotide bases. The target PMS2 genomic DNA or cDNA and the oligonucleotide probe can be contacted with each other under conditions sufficiently stringent such that the genetic variant can be distinguished from the wild-type PMS2 gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular genetic variant.

In one embodiment, the relative abundance of particular exons or introns in the PMS2 gene, and thus the presence or absence of deletions within the PMS2 gene, may be determined using Multiplex Ligation-dependent Probe Amplification (MLPA) assays. In MLPA assays, the presence or absence of a particular exon or intron is determined by the presence or absence of a PCR product amplified from two ligated oligonucleotides, respectively. The two oligonucleotides are designed to comprise a PMS2 sequence such that if the two oligonucleotides hybridize to the complementary strand of a PMS2 nucleic acid, the 5′ end of one of the oligonucleotides will be adjacent to the 3′ end of the other oligonucleotide, such that a ligase may covalently bind the two oligonucleotides together. The oligonucleotides are additionally designed to comprise a primer hybridization sequence on each, such that the ligated product will be amplified in the presence of the appropriate primers in a PCR reaction. The oligonucleotide sequences may additionally contain a stuffer sequence to ensure that any resulting PCR products will have a unique size. In an MLPA assay, the oligonucleotides are mixed with a nucleic acid sample in conditions to promote hybridization of the oligonucleotides to homologous nucleotide sequences in the sample. Afterwards a ligase is added, to ligate any sets of oligonucleotides that have hybridized to a template in such a way that the ligase may covalently bond the two oligonucleotides. Finally the product of the ligation reaction is subjected to PCR, such that any ligated fragments will result in a PCR fragment. By designing oligonucleotides that result in different sized PCR products, multiplex reactions are possible. The relative amounts of PCR product derived from the reactions will indicate the relative amount of the PMS2 sequences in the sample to which the oligonucleotides hybridized. MLPA is further described in Schouten J P et al. (2002) Relative quantification of 40 nucleic acid sequences by multiplex ligation-dependent probe amplification Nucleic Acids Res 30, e57.

Another useful technique that is gaining increased popularity is mass spectrometry. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997). In another example, primers can be designed based on either mutant or wild-type PMS2 gene intronic or exonic sequences such that the primers have the nucleotide sequences adjacent to and flanking a deletion locus identified in accordance with the present invention. PCR amplification on a patient sample is carried out using the primers. Mass spectrometry is then performed on the PCR product.

In addition, the microchip or microarray technologies are also applicable to the detection method of the present invention. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations particularly SNPs. The microchip technologies combined with computerized analysis tools allow fast screening in a large scale. The adaptation of the microchip technologies to the present invention will be apparent to a person of skill in the art apprised of the present disclosure. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

As is apparent from the above survey of the suitable detection techniques, it may or may not be necessary to amplify the target DNA, i.e., the PMS2 genomic DNA or cDNA sequence to increase the number of target DNA molecules, depending on the detection techniques used. For example, most PCR-based techniques combine the amplification of a portion of the target and the detection of mutations. PCR amplification is well known in the art and is disclosed in U.S. Pat. Nos. 4,683,195 and 4,800,159, both of which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2^{nd} ed., Cold Spring Harbor Laboratory, Cold Spring Harbor, N.Y., 1989. However, even with scarce samples, many sensitive techniques have been developed in which genetic variations can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theon. Biol., 187:273-284 (1997).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations between fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995). Additionally, the Invader assay and the rolling circle amplification technique may also be used. See e.g. Lyamichev et al., Nat. Biotechnol., 17:292-296 (1999); Lizardi et al., Nature Genetics, 19:225-232 (1998).

In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a genetic variant in the PMS2 gene of a particular individual should be apparent to a skilled artisan apprised of this disclosure.

Protein-based detection techniques may also prove to be useful, especially when the genetic variant causes amino acid substitutions or deletions or insertions that affect the protein primary, secondary or tertiary structure. To detect the amino acid variations, protein sequencing techniques may be used. For example, a PMS2 protein or fragment thereof can be synthesized by recombinant expression using a PMS2 DNA fragment isolated from an individual to be tested. Preferably, a PMS2 cDNA fragment of no more than 100 to 150 base pairs encompassing the polymorphic locus to be determined is used. The amino acid sequence of the peptide can then be determined by conventional protein sequencing methods. Alternatively, the recently developed HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected therefrom is analyzed. See Gatlin et al., Anal. Chem., 72:757-763 (2000).

Other useful protein-based detection techniques include immunoaffinity assays based on antibodies selectively immunoreactive with mutant PMS2 proteins according to the present invention. Such antibodies may react specifically with epitopes comprising the polypeptide fragments spanning the junction regions of PMS2 proteins that correspond to deletion loci in the mutant PMS2 mRNAs transcribed from the mutant PMS2 genomic DNAs of the present invention. Methods for producing such antibodies are described above in detail. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well known antibody-based techniques can also be used including, e.g., enzyme-linked immunosorbent assay (ELISA), radioimmunoassay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530, both of which are incorporated herein by reference.

It is noted that heterozygotes of the PMS2 genetic variants of the present invention are predisposed to cancer such as Lynch syndrome associated cancers. That is, as long as an individual has one chromosome containing a PMS2 genetic variant of the present invention, there is an increased likelihood of a Lynch syndrome associated cancer in the individual.

Thus, various techniques can be used in genotyping a PMS2 gene of an individual to determine, in the individual, the presence or absence of a PMS2 genetic variant selected from the group consisting of the deletions in Table 1. Typically, once the presence or absence of a PMS2 genetic variant of the present invention is determined, the result can be cast in a communicable form that can be communicated to the individual patient. Such a form can vary and can be tangible or intangible. The result with regard to the presence or absence of a PMS2 genetic variant of the present invention in the individual tested can be embodied in descriptive statements, diagrams, photographs, charts, images or any other visual forms. For example, images of gel electrophoresis of PCR products can be used in explaining the results. Diagrams showing where a deletion occurs in an individual's PMS2 gene are also useful in communicating the test results. The statements and visual forms can be recorded on a tangible media such as papers, computer readable media such as floppy disks, compact disks, etc., or on an intangible media, e.g., an electronic media in the form of e-mail, or on a preferably secured website on the internet or an intranet. In addition, the result with regard to the presence or absence of a PMS2 genetic variant of the present invention in the individual tested can also be recorded in a sound form and transmitted through any suitable media, e.g., analog or digital cable lines, fiber optic cables, etc., via telephone, facsimile, wireless mobile phone, internet phone and the like.

The present invention also provides kits for practicing the genotyping methods described above. The kits may include a carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting nucleotide or amino acid variants discovered in accordance with the present invention using the above-discussed detection techniques.

In one embodiment, the detection kit includes one or more oligonucleotides useful in detecting the genetic variants in PMS2 gene sequence in accordance with the present invention. Preferably, the oligonucleotides are designed such that they are specific to a PMS2 nucleic acid variant of the present invention under stringent conditions. That is, the oligonucleotides should be designed such that it can be used in distinguishing one genetic variant. Thus, the oligonucleotides can be used in mutation-detecting techniques such as allele-specific oligonucleotides (ASO), allele-specific PCR, TaqMan-based quantitative PCR, chemiluminescence-based techniques, molecular beacons, and improvements or derivatives thereof, e.g., microchip technologies.

In another embodiment of this invention, the kit includes one or more oligonucleotides suitable for use in detecting techniques such as ARMS, oligonucleotide ligation assay (OLA), and the like. For example, the oligonucleotides in this embodiment include a PMS2 gene sequence immediately 5′ upstream from a deletion locus to be analyzed. The 3′ end nucleotide of the oligo is the first nucleotide on the 3′ side of the deletion locus.

In another embodiment, the kit includes two or more oligonucleotides suitable for use in detecting the relative abundance of an exon or intron using MLPA.

The oligonucleotides in the detection kit can be labeled with any suitable detection marker including but not limited to, radioactive isotopes, fluorophores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with a PMS2 protein variant of the present invention. Methods for producing and using such antibodies have been described above in detail.

Various other components useful in the detection techniques may also be included in the detection kit of this invention. Examples of such components include, but are not limited to, DNA polymerase, reverse transcriptase, deoxyribonucleotides, dideoxyribonucleotides other primers suitable for the amplification of a target DNA or mRNA sequence, RNase A, mutS protein, and the like. In addition, the detection kit preferably includes instructions on using the kit for detecting genetic variants in PMS2 gene sequences, particularly the genetic variants of the present invention.

### 7. Cell and Animal Models

In yet another aspect of the present invention, a cell line and a transgenic animal carrying a PMS2 nucleic acid variant in accordance with the present invention are provided. The cell line and transgenic animal can be used as model systems for studying cancers and testing various therapeutic approaches in treating cancers, including Lynch syndrome associated cancers.

To establish the cell line, cells expressing the mutant PMS2 protein can be isolated from an individual carrying the genetic variants. The primary cells can be transformed or immortalized using techniques known in the art. Alternatively, normal cells expressing a wild-type PMS2 protein or other type of genetic variants can be manipulated to replace the entire endogenous PMS2 gene with a PMS2 nucleic acid variant of the present invention, or simply to introduce mutations into the endogenous PMS2 gene. The genetically engineered cells can further be immortalized.

A more valuable model system is a transgenic animal. A transgenic animal can be made by replacing its endogenous PMS2 gene ortholog with a human PMS2 nucleic acid variant of the present invention. Alternatively, deletions can be introduced into the endogenous animal PMS2 gene ortholog to simulate the PMS2 alleles discovered in accordance with the present invention. Techniques for making such transgenic animals are well known and are described in, e.g., Capecchi, et al., Science, 244:1288 (1989); Hasty et al., Nature, 350:243 (1991); Shinkai et al., Cell, 68:855 (1992); Mombaerts et al., Cell, 68:869 (1992); Philpott et al., Science, 256:1448 (1992); Snouwaert et al., Science, 257:1083 (1992); Donehower et al., Nature, 356:215 (1992); Hogan et al., Manipulating the Mouse Embryo; A Laboratory Manual, 2^{nd} edition, Cold Spring Harbor Laboratory Press, 1994; and U.S. Pat. Nos. 5,800,998, 5,891,628, and 4,873,191, all of which are incorporated herein by reference.

The cell line and transgenic animal are valuable tools for studying the mutant PMS2 genes, and in particular for testing in vivo the compounds identified in the screening method of this invention and other therapeutic approaches as discussed above. As is well known in the art, studying drug candidates in a suitable animal model before advancing them into human clinical trials is particularly important because not only can efficacy of the drug candidates can be confirmed in the model animal, but the toxicology profiles, side effects, and dosage ranges can also be determined. Such information is then used to guide human clinical trials.

### 8. Methods of Detecting a Mutation

In accordance with another aspect of the invention, methods of detecting a mutation in PMS2 nucleic acid are disclosed. In a specific embodiment, the method comprises:
a. analyzing PMS2 nucleic acid in a sample obtained from an individual; and
b. detecting any one of the mutations in Table 1.

Any method of analyzing a nucleic acid sample and detecting a mutaition, as described herein (and in particular in the section entitled Genotyping), may be utilized.

In related embodiments, the method specifically comprises detecting a deletion of exons 1-11. In related embodiments, the method specifically comprises detecting a deletion of exons 6-15. In related embodiments, the method specifically comprises detecting a deletion of exons 7-11. In related embodiments, the method specifically comprises detecting a deletion of exon 11. In related embodiments, the method specifically comprises detecting a deletion of exons 11-12. In related embodiments, the method specifically comprises detecting a deletion of the entire PMS2 gene. In related embodiments, the method specifically comprises detecting a deletion of exons 1-5. In related embodiments, the method specifically comprises detecting a deletion of exons 1-9. In related embodiments, the method specifically comprises detecting a deletion of exons 1-10. In related embodiments, the method specifically comprises detecting a deletion of exons 5-9. In related embodiments, the method specifically comprises detecting a deletion of exons 6-9. In related embodiments, the method specifically comprises detecting a deletion of exon 8. In related embodiments, the method specifically comprises detecting a deletion of exons 9-10. In related embodiments, the method specifically comprises detecting a deletion of exon 10.

In some embodiments, the method of detecting further comprises obtaining a sample from an individual. The sample may be obtained by any means known in the art, and as specifically described herein. The sample may be any biological sample which comprises DNA, including skin, saliva, urine, blood, buccal, tear, hair, and nail clippings.

### 9. Methods of Diagnosing an Individual with Lynch Syndrome

In accordance with another aspect of the invention, methods for diagnosing an individual with Lynch Syndrome are disclosed. In a specific embodiment, the method comprises:
a. analyzing PMS2 nucleic acid in a sample obtained from an individual;
b. detecting any one of the mutations in Table 1; and
c. diagnosing the individual with Lynch Syndrome based at least in part on detecting the mutation.

Any method of analyzing a nucleic acid sample and detecting a mutaition, as described herein (and in particular in the section entitled Genotyping), may be utilized.

In related embodiments, the method specifically comprises detecting a deletion of exons 1-11. In related embodiments, the method specifically comprises detecting a deletion of exons 6-15. In related embodiments, the method specifically comprises detecting a deletion of exons 7-11. In related embodiments, the method specifically comprises detecting a deletion of exon 11. In related embodiments, the method specifically comprises detecting a deletion of exons 11-12. In related embodiments, the method specifically comprises detecting a deletion of the entire PMS2 gene. In related embodiments, the method specifically comprises detecting a deletion of exons 1-5. In related embodiments, the method specifically comprises detecting a deletion of exons 1-9. In related embodiments, the method specifically comprises detecting a deletion of exons 1-10. In related embodiments, the method specifically comprises detecting a deletion of exons 5-9. In related embodiments, the method specifically comprises detecting a deletion of exons 6-9. In related embodiments, the method specifically comprises detecting a deletion of exon 8. In related embodiments, the method specifically comprises detecting a deletion of exons 9-10. In related embodiments, the method specifically comprises detecting a deletion of exon 10.

In some embodiments, the method of detecting further comprises obtaining a sample from an individual. The sample may be obtained by any means known in the art, and as specifically described herein. The sample may be any biological sample which comprises DNA, including skin, saliva, urine, blood, buccal, tear, hair, and nail clippings.

In some embodiments, the step of diagnosing the individual with Lynch Syndrome based at least in part on detecting the mutation consists of diagnosing the individual with Lynch Syndrome based entirely on detecting the mutation. In other embodiments, the step of diagnosing the individual with Lynch Syndrome based at least in part on detecting the mutation comprises diagnosing the individual based, in part, on at least one additional clinical feature of the individual. In some embodiments, the at least one additional clinical features may comprise a personal or family history of cancer.

### 10. Methods of Prophylactically Treating an Individual for a Lynch Syndrome Associated Cancer

In accordance with another aspect of the invention, methods Methods of prophylactically treating an individual for a Lynch Syndrome associated cancer are disclosed. In a specific embodiment, the method comprises:
a. analyzing PMS2 nucleic acid in a sample obtained from an individual;
b. detecting any one of the mutations in Table 1; and
c. prophylactically treating the individual based at least in part on detecting the mutation.

Any method of analyzing a nucleic acid sample and detecting a mutaition, as described herein (and in particular in the section entitled Genotyping), may be utilized.

In related embodiments, the method specifically comprises detecting a deletion of exons 1-11. In related embodiments, the method specifically comprises detecting a deletion of exons 6-15. In related embodiments, the method specifically comprises detecting a deletion of exons 7-11. In related embodiments, the method specifically comprises detecting a deletion of exon 11. In related embodiments, the method specifically comprises detecting a deletion of exons 11-12. In related embodiments, the method specifically comprises detecting a deletion of the entire PMS2 gene. In related embodiments, the method specifically comprises detecting a deletion of exons 1-5. In related embodiments, the method specifically comprises detecting a deletion of exons 1-9. In related embodiments, the method specifically comprises detecting a deletion of exons 1-10. In related embodiments, the method specifically comprises detecting a deletion of exons 5-9. In related embodiments, the method specifically comprises detecting a deletion of exons 6-9. In related embodiments, the method specifically comprises detecting a deletion of exon 8. In related embodiments, the method specifically comprises detecting a deletion of exons 9-10. In related embodiments, the method specifically comprises detecting a deletion of exon 10.

In some embodiments, the method of detecting further comprises obtaining a sample from an individual. The sample may be obtained by any means known in the art, and as specifically described herein. The sample may be any biological sample which comprises DNA, including skin, saliva, urine, blood, buccal, tear, hair, and nail clippings.

In some embodiments, the step of prophylactically treating the individual based at least in part on detecting the mutation comprises treating the individual with a medicament. In some embodiments, the step of prophylactically treating the individual based at least in part on detecting the mutation comprises performing surgery on the individual. In some embodiments, the step of prophylactically treating the individual based at least in part on detecting the mutation comprises surgically removing a tissue from the individual. In some embodiments, the step of prophylactically treating the individual based at least in part on detecting the mutation comprises performing more aggressive surveillance on the individual.

, the method comprises treating the individual with a medicament or other intervention to reduce the likelihood of the individual developing a Lynch Syndrome associated cancer. In one embodiment, the method comprises performing surgery on the individual to reduce the likelihood of the individual developing a Lynch Syndrome associated cancer.

Although the foregoing invention has been described in some detail by way of illustration and example for purposes of clarity of understanding, it will be obvious that certain changes and modifications may be practiced within the scope of the appended claims.

All publications mentioned in the specification are indicative of the level of those skilled in the art to which this invention pertains. All publications and patent applications are herein incorporated by reference to the same extent as if each individual publication or patent application was specifically and individually indicated to be incorporated by reference.

### 11. EXAMPLES

### Example 1

### Methods

After informed consent was obtained by a healthcare provider, peripheral blood or buccal mouthwash samples were collected by the healthcare provider from patients with a personal and/or family history suggestive of Lynch syndrome or hereditary colon cancer. Genomic DNA was extracted from each sample for subsequent genetic analysis. Comprehensive analysis, which includes concurrent sequencing and large rearrangement analyses for DNA mutations in the MLH1, MSH2, MSH6, PMS2, and EPCAM genes, was performed.

For sequencing analysis, genomic DNA was directly amplified utilizing the Polymerase Chain Reaction (PCR) to generate exon-specific amplicons for MLH1, MSH2, MSH6, EPCAM, and portions of the PMS2 gene. Due to the presence of pseudogenes, long range PCR with a nested PCR approach was utilized for the remaining portions of PMS2, allowing for the detection of sequencing mutations along the entire PMS2 coding region. PCR products were sequenced in the forward and reverse orientations using fluorescent dye-labeled sequencing primers, and resulting chromatograms were compared to a known wild type sequence.

MLH1 and MSH2 large rearrangement analysis (CART) was performed using a quantitative multiplex PCR assay, which detects copy number variations indicative of a deletion or insertion. PMS2, EPCAM, and MSH6 large rearrangement analyses were performed using Multiplex Ligation-dependent Probe Amplification (MLPA) assays.

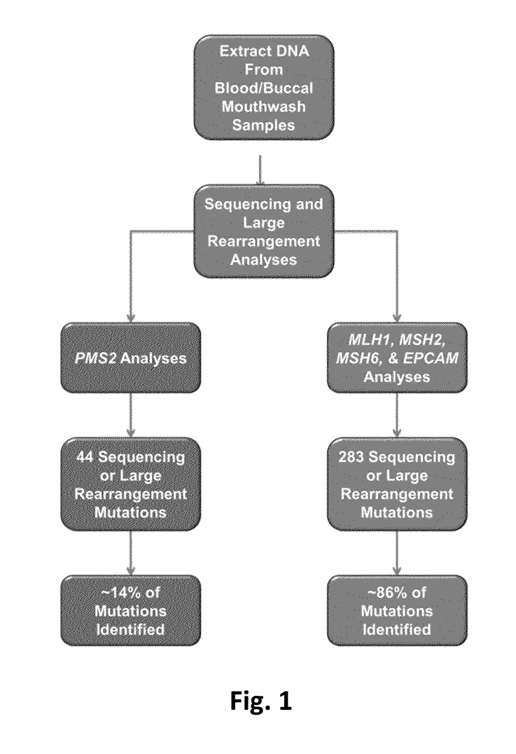

In addition to concurrent genetic analysis of the MLH1, MSH2, MSH6, PMS2, and EPCAM genes, some patients opted for a more targeted gene approach, allowing for identification of additional sequencing and large rearrangement mutations in the PMS2 gene. For an overview of the study, see FIG. 1.

Results

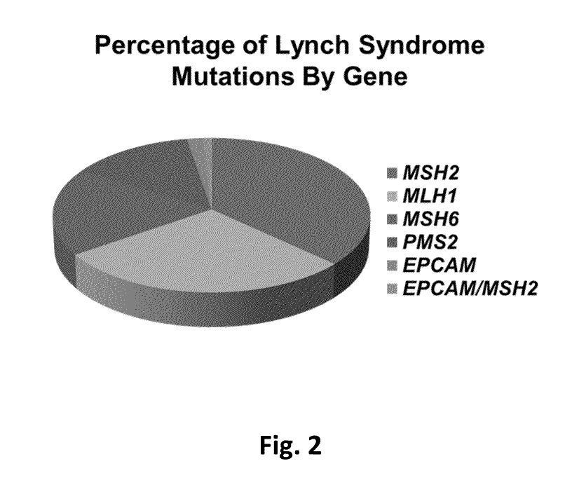

Frequency of PMS2 Mutations—Molecular genetic testing of a patient cohort for Lynch syndrome was performed in our clinical diagnostic laboratory. Testing included concurrent sequencing and large rearrangement analyses of the PMS2, MLH1, MSH2, MSH6, and EPCAM genes. 327 disease-associated mutations were identified within this patient population. 44/327 (14%) of the identified mutations were within the PMS2 gene (FIG. 2). This observed prevalence of PMS2 mutations is at the high end of previously reported estimates of PMS2 mutation frequency.⁵⁻⁷

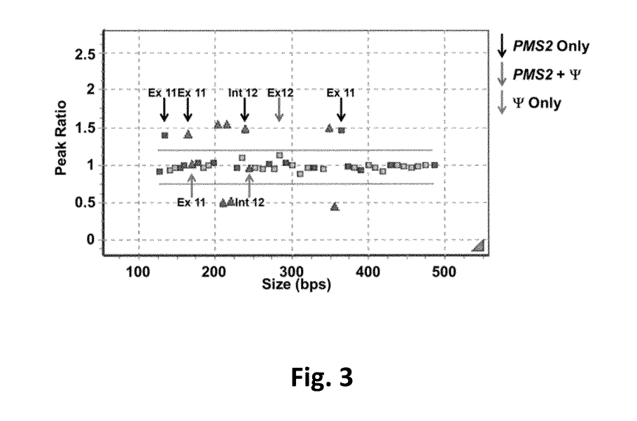

In addition to patients receiving comprehensive testing for mutations in PMS2, MLHJ, MSH2, MSH6, and EPCAM, some patients received more targeted genetic analysis for mutations in PMS2 alone or PMS2 in combination with one or more genes. These analyses have identified 37 unique sequencing and 14 unique large rearrangement PMS2 mutations (FIG. 3). This corresponds to a PMS2 mutation profile of ˜73% sequencing versus ˜27% large rearrangement mutations.

Analysis of PMS2 Pseudogene Regions—Analysis of the PMS2 gene is complicated by the presence of multiple pseudogenes which span exons 1-5, 9, and 11-15. For sequencing analysis, long range PCR with subsequent nested PCR amplification and sequencing can be utilized to generate coding gene-specific DNA sequences. This allows for sequencing analysis of the entire PMS2 coding region.

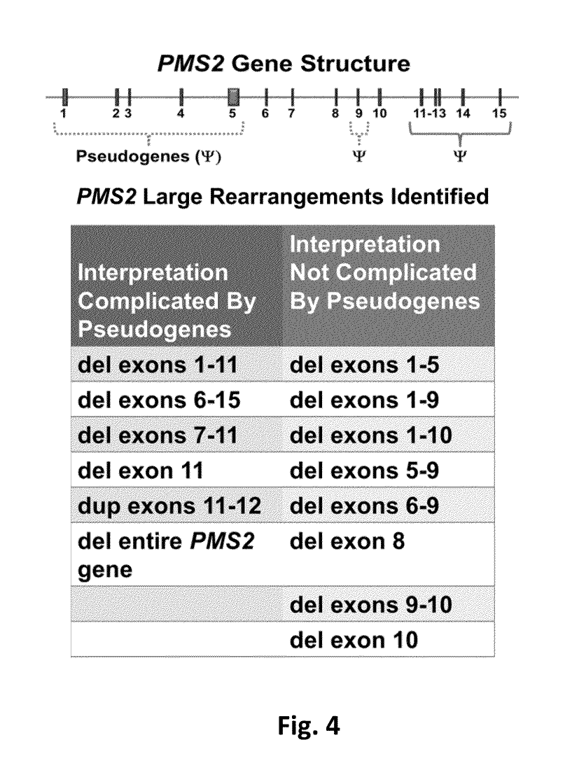

Due to the presence of multiple single nucleotide polymorphisms in or near exons 1-5 and 9, it is usually possible to distinguish the PMS2 coding gene from confounding pseudogenes, allowing for MLPA large rearrangement analysis to be more readily performed for exons 1-10. However, the pseudogene spanning exons 11-15 bears strong homology to the coding gene, and gene conversions events are common. Thus, the interpretation of MLPA test results within this region may require additional confirmatory analyses such as long range PCR and sequencing of both coding gene and pseudogene regions of interest. Understanding these complexities, we have identified multiple large rearrangements within PMS2 coding regions known to have interfering pseudogenes (FIG. 4, Table 1).

Conclusions

PMS2 gene mutations are responsible for ˜14% of Lynch syndrome cases for which causative mutations were identified.

A PMS2 mutation profile of ˜73% sequencing versus ˜27% large rearrangement mutations was identified.

PMS2 pseudogenes increase the complexity of genetic analysis, but sequencing and large rearrangements were detected.

The identification and interpretation of PMS2 large rearrangements within exons 11-15 is complicated by the presence of a highly homologous pseudogene and gene conversion. However, additional analyses, as described herein, can clarify these results.

Comprehensive diagnostic genetic testing for Lynch syndrome should include sequencing and large rearrangement analyses of the PMS2 gene.

What is claimed is:

1. A method for predicting a predisposition to cancer in a patient, comprising:
detecting a deletion in the PMS2 gene, wherein said deletion is selected from:
(1) a deletion of exons 1-11;
(2) a deletion of exons 6-15;
(3) a deletion of exons 7-11;
(4) a deletion of exon 11;
(5) a deletion of exons 11-12;
(6) a deletion of the entire PMS2 gene;
(7) a deletion of exons 1-5;
(8) a deletion of exons 1-9;
(9) a deletion of exons 1-10;
(10) a deletion of exons 5-9;
(11) a deletion of exons 6-9;
(12) a deletion of exon 8;
(13) a deletion of exons 9-10;
(14) a deletion of exon 10;
wherein the presence of the deletion would indicate a predisposition to cancer.

2. The method of claim 1 wherein the detection step comprises analysis of PMS2 genomic DNA.

3. The method of claim 2 wherein the analysis of PMS2 genomic DNA comprises amplifying a region of genomic DNA in which the deletion occurs.

4. The method of claim 2 wherein the analysis of PMS2 genomic DNA comprises hybridizing a nucleic acid probe to a region of genomic DNA in which the deletion occurs.

5. The method of claim 1 wherein the detection step comprises analysis of PMS2 cDNA.

6. The method of claim 5 wherein the analysis of PMS2 cDNA comprises amplifying a region of cDNA in which the deletion occurs.

7. The method of claim 5 wherein the analysis of PMS2 cDNA comprises hybridizing a nucleic acid probe to a region of cDNA in which the deletion occurs.

8. The method of claim 1 wherein the detection step comprises analysis of a PMS2 polypeptide.

9. The method of claim 8 wherein the analysis of a PMS2 polypeptide comprises determining whether the polypeptide is truncated.

10. The method of claim 8 wherein the analysis of a PMS2 polypeptide comprises contacting the polypeptide with an antibody.

11. The method of claim 1, wherein the said deletion is selected from:
(1) a deletion of exons 1-11;
(2) a deletion of exons 6-15;
(3) a deletion of exons 7-11;
(4) a deletion of exon 11;
(5) a deletion of exons 11-12;
(6) a deletion of the entire PMS2 gene.

12. The method of claim 1, wherein the deletion is a deletion of exons 1-11.

13. The method of claim 1, wherein the deletion is a deletion of exons 6-15;

14. The method of claim 1, wherein the deletion is a deletion of exons 7-11;

15. The method of claim 1, wherein the deletion is a deletion of exon 11;

16. The method of claim 1, wherein the deletion is a deletion of exons 11-12;

17. The method of claim 1, wherein the deletion is a deletion of the entire PMS2 gene.

18. The method of claim 2, wherein the step of detecting the deletion comprises performing a Multiplex Ligation-dependent Probe Amplification (MLPA) assay.

19. A method of prophylactically treating an individual for a Lynch Syndrome associated cancer, comprising:
a. analyzing PMS2 nucleic acid in a sample obtained from the individual;
b. detecting a deletion in the PMS2 gene, wherein said deletion is selected from:
(1) a deletion of exons 1-11;
(2) a deletion of exons 6-15;
(3) a deletion of exons 7-11;
(4) a deletion of exon 11;
(5) a deletion of exons 11-12;
(6) a deletion of the entire PMS2 gene;
(7) a deletion of exons 1-5;
(8) a deletion of exons 1-9;
(9) a deletion of exons 1-10;
(10) a deletion of exons 5-9;
(11) a deletion of exons 6-9;
(12) a deletion of exon 8;
(13) a deletion of exons 9-10;
(14) a deletion of exon 10; and
c. prophylactically treating the individual based at least in part on detecting the deletion.
